(19) United States
(12) Patent Application Publication
(10) Pub. No.: US 2024/0117310 A1
(43) Pub. Date: Apr. 11, 2024

(54) Human Cross-Presenting CD141+CLEC9A+ Dendritic Cells, Methods of Producing the Same from Mobilized Peripheral Blood CD34+ Hematopoietic Stem Cells and Methods of Use

(71) Applicant: Duke University, Durham, NC (US)

(72) Inventors: Smita NAIR, Durham, NC (US); Adam SWARTZ, Durham, NC (US)

(21) Appl. No.: 18/468,526

(22) Filed: Sep. 15, 2023

Related U.S. Application Data
(60) Provisional application No. 63/406,847, filed on Sep. 15, 2022.

(51) Int. Cl. C12N 5/0784; A61K 39/00; A61K 39/395

(52) U.S. Cl. CPC C12N 5/0639; A61K 39/3955; A61K 39/4615; A61K 2039/5154; A61K 2039/575; C12N 2501/22; C12N 2501/26; C12N 2506/11

(57) ABSTRACT

The present disclosure describes systems and methods for in vitro differentiation of human cross-presenting CD141⁺CLEC9A⁺ dendritic cells from mobilized peripheral blood CD34⁺ hematopoietic stem cells. The dendritic cells may further comprise an antigen or nucleic acid encoding an antigen. Methods of using the cells are also provided.

### CROSS-REFERENCE TO RELATED APPLICATIONS

This application claims priority to U.S. Provisional Application No. 63/406,847 filed on Sep. 15, 2022, the contents of which are incorporated by reference in their entireties.

### STATEMENT REGARDING FEDERALLY SPONSORED RESEARCH

This invention was made with government support under grant no. W81XWH-20-1-0627 awarded by the US Department of Defense (DOD). The government has certain rights in the invention.

### REFERENCE TO AN ELECTRONIC SEQUENCE LISTING

The contents of the electronic sequence listing (155554.00705.xml; Size: 1,880 bytes; and Date of Creation: Sep. 12, 2023) is herein incorporated by reference in its entirety.

### BACKGROUND

Dendritic cells (DCs) are professional antigen presenting cells that serve as the sentinels of the immune system by surveying the environment for “non-self” material and alerting other cells of danger. Under healthy conditions, DCs exist in an immature state where they continuously sample cell-endogenous and exogenous antigen, which are processed through specialized antigen-presentation pathways and presented to CD8⁺ and CD4⁺ T lymphocytes. Upon detection of damaged cells or pathogen-associated motifs by pattern recognition receptors, DCs signal to other cells through the elaboration of various immune-stimulating cytokines and upregulation of several molecules that enhance their T-cell stimulating capabilities—a process known as maturation. Mature DCs are uniquely equipped to be potent activators of naïve T cells. This distinctive ability to bridge the innate and adaptive arms of the immune system makes DCs desirable targets for immunotherapy.

All DCs arise from bone marrow-resident progenitors produced from hematopoietic stem cells. Historically, human DCs have been divided into 3 major subsets: plasmacytoid DCs, conventional type 2 DCs (cDC2), and conventional type 1 DCs (cDC1). Plasmacytoid DCs (pDCs), which can originate from both the common myeloid and common lymphoid progenitor, are typified by the high expression of the toll-like receptors TLR7 and TLR9. While MoDCs remain the most common DC type used in clinical studies, several investigators evaluating natural DC vaccines have demonstrated that vaccination with natural DCs is safe, well-tolerated, and feasible despite the low frequency of DCs in the blood (<1% of circulating PBMCs). Accordingly, there is a remaining need in the art for a method for producing high numbers of CD141⁺Irf8⁺ human DCs in vitro.

### SUMMARY

The present disclosure provides compositions of cDC1 cells and methods of making and using the same. One aspect of the invention provides a population of in vitro or ex vivo derived dendritic cells, comprising at least 1×10⁶ dendritic cells/ml, wherein the dendritic cells are CD141⁺CLEC9A⁺.

A second aspect of the present invention provides a population of in vitro or ex vivo derived dendritic cells, wherein the dendritic cells are CD141⁺CLEC9A⁺ and wherein the dendritic cells comprise an antigen or nucleic acid added to the cells in vitro or ex vivo. In some embodiments the antigen is a peptide, and in some embodiments the antigen is an mRNA polynucleotide or a construct encoding an mRNA polynucleotide. In some embodiments the antigen is a viral, bacterial, fungal, or tumor antigen.

In some embodiments the population of in vitro or ex vivo derived dendritic cells, activate CD8⁺ T cells. In some embodiments, the population comprises at least 1×10⁷ dendritic cells. In some embodiments the dendritic cells are also Irf8⁺ and/or TLR3⁺ and are human dendritic cells.

Another aspect of the invention provides a method of treating a subject having a disease, wherein the disease is a viral infection, bacterial infection, fungal infection or cancer, or a disease wherein a Th1 immune response would be beneficial to the subject comprising administering to the subject the population of cells described herein. In some embodiments the population of cells are administered intratumorally.

Another aspect of the present invention provides a method of treating a subject having a disease, wherein the disease is a viral infection, bacterial infection, fungal infection or cancer, or a disease wherein a Th1 immune response would be beneficial to the subject comprising administering to the subject the population of cells described herein, wherein the antigen is a peptide, mRNA or construct encoding an mRNA. In some embodiments, the population of cells are administered intravenously, intramuscularly, subcutaneously, intradermally or intratumorally. In some embodiment, the population of dendritic cells are differentiated from CD34⁺ hematopoietic stem cells. In some embodiments, the CD34⁺ hematopoietic stem cells are differentiated in a cell culture media comprising FLT3L and GM-CSF. In some embodiments, the population of dendritic cells are produced on sterile untreated polystyrene culture plates.

In some embodiments of the present invention, CD34⁺ hematopoietic stem cells are mobilized and collected from the subject, differentiated into the population of dendritic cells prior to being administered to the subject to provide a population of autologous dendritic cells to the subject. In some embodiments, the population of dendritic cells are administered more than once to the subject.

Another aspect of the present disclosure provides a method of enhancing an immune response in a subject, comprising administering to a subject a population of cells described herein.

In some embodiments, the cells comprise and antigen wherein the antigen or nucleic acid is derived from a virus, bacteria, fungus, or tumor. In some embodiments, the immune response is directed to a viral infection, bacterial infection, fungal infection, or cancer. In some embodiments, the immune response is a Th1 immune response. In some embodiments, the subject is human.

In some embodiments the methods provided herein additionally comprise administering immune checkpoint inhibitor therapy.

Another aspect of the present invention provides a method of generating a population of human CD141⁺CLEC9A⁺ dendritic cells comprising (a) harvesting or obtaining CD34⁺ hematopoietic stem cells from the peripheral blood of a subject; (b) culturing the CD34⁺ hematopoietic stem cells in non-tissue culture treated containers at a plating density of 1-3×10⁵ cells/cm² in cell culture media comprising FLT3L and GM-CSF to allow the cells to differentiate into cDC1 cells; and (c) collecting the CD141⁺ CLEC9A⁺ dendritic cells. In some embodiments, the subject was treated with G-CSF and/or Plerixafor to mobilize CD34⁺ hematopoietic stem cells into the peripheral blood at least one day prior to harvesting. In some embodiments, the subject is human. In some embodiments, the subject is in need of treatment with a cDC1 cell-based therapy. In some embodiments 50-250 ng/mL of human FLT3L and/or 1-20 ng/mL of human GM-CSF is added to the cell culture media in step (b). In some embodiments culturing in step (b) is for at least 6 days and no more than 21 days. In some embodiments, the cells harvested in step (c) are non-adherent or semi-adherent cells.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1. Timeline diagram illustrating the major steps of differentiating mobilized peripheral blood CD34⁺ HSCs into Irf8⁺ CD141⁺ DCs. Gating in flow cytometry plots shows the percentage of Irf8⁺ cells expressing intermediate and high levels of CD141, subgated from viable singlets.

FIG. 2. Representative flow cytometry histograms showing expression of DC markers on cells harvested on day 15 of the Basic Protocol, subgated from viable CD141^{HI} cells. Light gray histogram: FMO control; dark gray histogram: marker expression.

FIG. 3. In vitro CD141⁺CLECL9A⁺ DCs (cDC1) generated from CD34⁺ HSCs are superior to in vitro monocyte-derived DCs (MoDC) at cross presenting exogenous antigen to CD8⁺ T cells. 10⁴ in vitro CD141⁺CLECL9A⁺ DCs or MoDCs derived from an HLA*0201 donor were incubated with 10 ng/mL, 100 ng/mL, 1 μg/mL, or 10 μg/mL recombinant CMV protein for 3 h in a V-bottom plate and then washed thrice with sterile PBS. 5×10⁴ human CD8⁺ T cells reactive to the CMV HLA-A*0201 epitope NLVPMVATV (SEQ ID NO: 1) were added to DCs and incubated for 20 h. Supernatant was collected and analyzed for human IFNγ by ELISA. Statistics were performed using Student's T-test.

FIG. 4. In vitro CD141⁺ CLECL9A⁺ DCs (cDC1) generated from CD34⁺ HSCs are responsive to TLR ligand stimulation. 5×10⁴ in vitro cDC1 were stimulated with 10 μg/mL high molecular weight (HMW) polyinosinic-polycytidylic acid (PolyIC), 5 nM R848 (Resiquimod), 500 nM CpG ODN 2006, 500 ng/mL lipopolysaccharide (LPS), or left unstimulated for 24 h. Supernatant was collected and analyzed for various human inflammatory cytokines using ELISA. Collectively, these data suggest that in vitro cDC1 express functional TRL3, TLR7/8, and TLR4 but not TLR9.

### DETAILED DESCRIPTION

The present invention provides compositions of cDC1 cells and methods of making and using the same. One aspect of the present disclosure provides simplified or improved systems and methods for generating large numbers of cDC1-like cells in vitro or ex vivo from mobilized human peripheral blood CD34⁺ hematopoietic stem cells using FMS-like tyrosine kinase 3 ligand (FLT3L) and granulocyte macrophage colony-stimulating factor (GM-CSF). A distinguishing aspect of this protocol is the growth of cells without the need for any feeder cells including immortalized feeder cells and also that the cells are cultured on a non-tissue culture-treated surface rather than on a tissue culture-treated surface. The resulting CD11c⁺ DCs express high levels of cDC1-specific markers such as CD141, CLEC9A, TLR3, and several DC maturation markers. Furthermore, the cDC1s described herein are superior at exogenous antigen cross-presentation than the oft-used monocyte-derived DC. Compared to alternative differentiation methods, the present method generates large numbers of cDC1-like cells and can be useful for studying cDC1 immunobiology and clinical applications of this DC subset.

### Compositions:

One aspect of the present invention provides compositions of in-vitro or ex vivo derived dendritic cells. Dendritic cells (DCs) are antigen-presenting cells that capture, process, and present antigens to lymphocytes to initiate and regulate the adaptive immune response. DCs play a pivotal role in the innate detection of pathogens and the subsequent activation of the adaptive immune response. DCs initiate the adaptive response by presenting antigenic peptides on major histocompatibility complex (MHC) molecules to induce T cell activation and differentiation. DCs also secrete cytokines and growth factors that enhance and modulate immune responses. In addition to their role in activating naïve T cells, DCs are thought to play a critical role in guiding the differentiation of regulatory T cells as well as the development of T cell tolerance. As key sentinel cells, they reside throughout the body, particularly in lymphoid organs and at environmental interfaces such as the intestine and skin.

Most DCs arise through a common DC progenitor (CDP) that differentiates into conventional DC precursors (pre-cDCs) and plasmacytoid DC precursors (pre-pDCs) in a fms-like tyrosine kinase 3 (FLT3) ligand-dependent manner. E2-2-dependent pDC differentiation continues in the bone marrow, while the two conventional DCs emerge from pre-cDCs in peripheral lymphoid tissues. Additional monocyte-derived cells with DC-like properties (moDCs) can be generated during pathogen-initiated inflammation via the differentiation of monocytes.

Conventional, also called classical DC (cDC), have been divided into two subsets, cDC1 and cDC2 based on phenotypic markers and function. cDC2 cells engage in the general functions normally ascribed to the DC family, the priming of naïve CD4⁺ T cells through antigen presentation on MHC class II, and co-stimulation. cDC1 cells are specialized in cross-presentation, or the presentation of exogenous antigen on MHC class I to induce naive CD8⁺ T cells to acquire cytotoxic T lymphocyte (CTL) effector function. Consistent with their specialization, cDC1 and cDC2 cells also express the cytokines required to elicit CD4⁺ or CD8⁺ effector T cell functions. cDC2-driven helper T cell polarization leads to robust adaptive immune responses to extracellular pathogens, including microbial and helminth infections. The ability to cross-prime CD8⁺ T cells allows cDC1 to direct CTLs to respond to intracellular pathogens and tumors.

cDC1 cells are present at incredibly low prevalence in the blood. The median yield of blood DCs is 0.69% of PBMCs. Within this population, the median frequencies of pDCs, cDC2s, and cDC1s are 47.6%, 49.4%, and 2.6%, respectively. Thus, cDC1s make up less than 0.03% of human PBMCs. In contrast to the low proportion of cDC in the blood, monocytes make up ˜5-10% of circulating leukocytes, thus, millions of MoDCs can be generated from only a few milliliters of blood. However clinical outcomes with MoDC have been modest. MoDCs are transcriptionally and phenotypically distinct from naturally occurring DCs, generate inferior T cell proliferative responses compared to blood-derived DCs, and have suboptimal ability to migrate to draining lymph nodes, which may underlie their poor clinical performance. Given the superiority of cDC1s in generating CD8⁺ T-cell immunity, improved methods for generating cDC1 cells are greatly needed.

Murine cDC1 cells are identified by CD8 or CD103 depending on their resident tissue, and murine cDC2 cells are distinguished by their expression of CD11b in addition to common cDC markers. Humans cDC1 cells can be distinguished from cDC2 by their respective expression of BDCA1 and BDCA3, along with additional markers. Since MoDCs and some macrophages can be difficult to distinguish from cDCs, exclusion of classic monocyte/macrophage markers, such as Ly6C and F4/80, and differential expression of certain markers such as CD64, MAR-1, MerTK, and CD88, may help to distinguish cDCs from other cell types.

In some embodiments, the disclosed compositions express CD141 (also known as Thrombomodulin, THBD), and C-Type Lectin Domain Containing 9A (CLEC9A) on their cell surface. In some embodiments, the compositions may also express Interferon Regulatory Factor 8 (Irf8) and or Toll-like receptor 3 (TLR3), CD11c, human leukocyte antigen (HLA-DR), CD86 and CD40 on their cell surface.

In some embodiments, the composition may comprise at least 1×10⁶ dendritic cells/ml. The composition may comprise at least 1×10⁶ cells/ml, 2×10⁶ cells/ml, 4×10⁶ cells/ml, 8×10⁶ cells/ml, 1×10⁷ cells/ml, 2×10⁷ cells/ml, 3×10⁷ cells/ml or more and any amount in between.

In some embodiments, the disclosed in vitro derived dendritic cells comprise an antigen or nucleic acid. The antigen may comprise any protein, peptide, polysaccharide, lipid or nucleic acid which induces an immune response. The nucleic acid may encode a protein or peptide that may be antigenic. The nucleic acid may be an RNA or mRNA or may be DNA. The nucleic acid may also be encoded in a construct, wherein the in vitro derived dendritic cells comprises the construct. For example, a construct encoding an mRNA antigen, wherein the dendritic cells comprise the construct and expresses the mRNA to produce the antigen. The disclosed dendritic cells comprising an antigen may be generated by any means known in the art. By way of example and not limitation, antigens may be transfected directly, delivered with a cell-penetrating peptide or a construct or vector, such as a viral vector or liposome-based system can be used such that the cells express the nucleic acid or protein. Antigens may be electroporated, or antigens may be incubated in the presence of the in vitro derived dendritic cells for uptake and presentation on major histocompatibility complexes (MHC) on the dendritic cells.

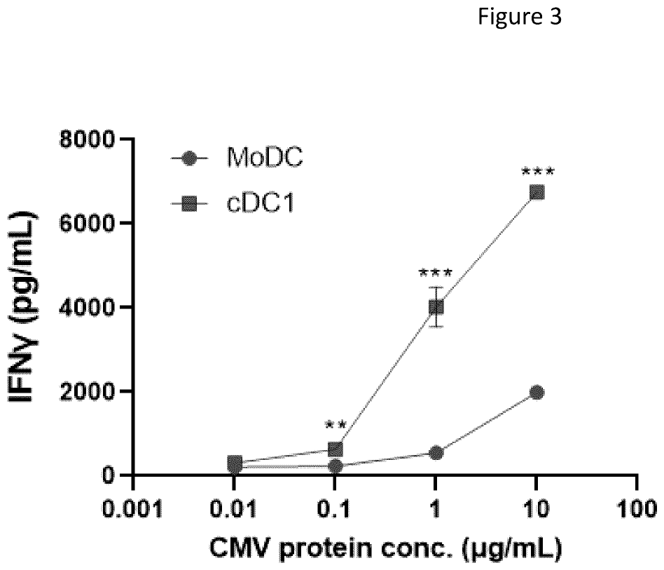

The dendritic cells disclosed herein may comprise an antigen derived from a pathogen. The pathogenic antigen may be of bacterial, fungal, parasitic or viral origin. The immune response activated by the dendritic cells provided herein may be particularly effective against intracellular pathogens such as viruses or bacterial or parasitic pathogens that live at least a portion of their infective period intracellularly. The antigen may also be a cancer-associated antigen or tumor antigen. In some embodiments, the in vitro derived dendritic cells comprising an antigen may activate CD8⁺ T cells or a Th1 immune response. Type 1 T helper (Th1) cells produce proinflammatory cytokines including, interferon-gamma, interleukin (IL)-2, and tumor necrosis factor (TNF)-beta, which activate immune cells and are responsible for cell-mediated immunity and phagocyte-dependent protective responses. Th1 cells activate macrophages natural killer cells, B cells and CD8⁺ T cells. By way of example, FIG. 3 demonstrates dendritic cells produced by the methods described herein, can cross present a cytomegalovirus antigen to CD8⁺ T cells and stimulate interferon γ production.

### Methods:

Another aspect of the disclosure provides a method of treating a subject having a disease or likely to have a disease. The method comprises administering any of the disclosed compositions. In some embodiments the disease is a viral, bacterial, fungal or parasitic infection. The disease may also be cancer. The subject may also have another disease or condition in which a Th1 immune response would be beneficial to the subject. The subject may be a human and the subject may have the disease or condition or be at high risk for developing the disease (i.e., be diagnosed with pre-cancer). The cells administered to the subject may be autologous. The cells may be made by the methods provided herein and are CD141⁺CLEC9A⁺. The dendritic cells may also be Irf8⁺ and/or TLR3⁺.

By way of example, and not limitation, the subject may have a cancer, wherein the cancer may be bladder cancer, bone cancer, brain cancer, breast cancer, cervical cancer, colon cancer, esophageal cancer, Gastric cancer, head & neck cancers, Hodgkin's lymphoma, leukemia, liver cancer, lung cancer, melanoma, mesothelioma, multiple myeloma, myelodysplastic syndrome, non-Hodgkin's lymphoma, ovarian cancer, pancreatic cancer, prostate cancer, rectal cancer, renal cancer, sarcoma, skin cancer, testicular cancer, thyroid cancer, uterine cancer and any other cancer or solid tumor.

The method of treating a subject having a disease may comprise administering the compositions described herein. As used herein, the term “administering” an agent, such as a therapeutic entity or composition described herein to an animal or cell, is intended to refer to dispensing, delivering or applying the substance to the intended target. In terms of the therapeutic agent composition, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. In some embodiments the composition is administered intravenously, intramuscularly, subcutaneously or intratumorally.

In some embodiments, the dendritic cells are administered more than once. Including but not limited to 2, 3, 4 or more times to a subject. Timing and dose of dendritic cell administration can be determined by one of skill in the art. Additional components may be administered in combination with the dendritic cells provided herein or produced by the methods provided herein. For example, the dendritic cells may be administered in combination with, before or after administration of other therapeutic compositions. The methods may further comprise administering a checkpoint inhibitor therapy or an anti-cancer therapeutic. Such therapeutics are known to those of skill in the art.

Another aspect of the present disclosure provides a method for enhancing an immune response in a subject. In some embodiments, the method comprises administering the dendritic cells described herein to induce an immune response in the subject. The dendritic cells may further comprise an antigen. The antigen may be derived from a pathogen including a viral, bacterial, or fungal antigen or a tumor antigen. The dendritic cells may enhance a Th1 immune response, including the production of proinflammatory proteins, tissue specific antigen presentation and an increased cell-mediated immune response. The enhanced immune response may also include an anti-tumor immune response. The subject may be a human and the subject may have the disease or condition or be at high risk for developing the disease (i.e., be diagnosed with pre-cancer). The cells administered to the subject may be autologous. The cells may be made by the methods provided herein and are CD141⁺CLEC9A⁺. The dendritic cells may also be Irf8⁺ and/or TLR3⁺.

The in vitro or ex vivo derived DC described herein may also include a suitable carrier or vehicle for administration. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Another aspect of the present invention provides methods of producing CD141⁺CLEC9A⁺ dendritic cells in vitro. The DC described herein may be differentiated from stem or progenitor cells including CD34⁺ hematopoietic stem cells. The CD34⁺ hematopoietic stem cells can be harvested or obtained from a commercial supplier of such cells. Suppliers of these cells are provided in the examples and are known to those of skill in the art. Alternatively, the CD34⁺ hematopoietic stem cells may be harvested directly from a subject. CD34⁺ hematopoietic stem cells may be collected by any means know in the art. In some embodiments, the CD34⁺ hematopoietic stem cells may be mobilized. Stem cell mobilization is a process whereby stem cells are stimulated out of the bone marrow space into the blood stream so they are available for collection. The cells may be mobilized by treating a subject with G-CSF and/or Plerixafor for at least one and up to 5 days prior to collection of the CD34⁺ cells from blood. The subject may be given up to 10 μg/kg/day for at least 3-5 days of G-CSF alone or up to 0.24 mg/kg of Plerixafor 1 day prior to harvesting the CD34⁺ cells or a combination thereof. Those of skill in the art may use other methods available to mobilize CD34⁺ hematopoietic cells and to harvest and select for those cells. In the Examples, these cells were obtained commercially, and those cells were obtained via mobilization with G-CSF followed by leukapheresis and positive immunomagnetic separation of CD34⁺ cells. The cells can then be preserved, frozen, stored or differentiated in-vitro. CD34⁺ cells can be mobilized by any means know in the art. In some embodiments, the CD34⁺ stem cells may be mobilized by colony stimulating factors (CSFs).

In some embodiments, CD34⁺ cells are differentiated into in-vitro derived cDC1 cells by culturing the cells with Fms-related tyrosine kinase 3 ligand (FLT3L) and granulocyte-macrophage colony-stimulating factor (GM-CSF). FLT3L may be used at concentrations in the range from about 50 ng/mL to about 250 ng/mL, 100 ng/mL to 200 ng/mL and 150 ng/mL was used in the examples. GM-CSF may be used at a concentration in a range from about 1 ng/mL to about 20 ng/mL, 5 ng/mL to 15 ng/mL or the 10 ng/mL used in the examples.

CD141⁺CLEC9A⁺ cDC1 cells may be differentiated in serum free media or media that comprises allogenic serum. For example, human CD141⁺CLEC9A⁺ cDC1 cells may be produced with media comprising human AB serum, or human serum albumin. CD141⁺CLEC9A⁺ cDC1 cells may also be produced in serum free media, including, but not limited to AIM-V, X-VIVO 15, RPMI 1640 and DMEM.

In some embodiments, the CD34⁺ cells may be differentiated into CD141⁺ CLEC9A⁺ cells in about 6 to about 10 days, or preferably 8 days. CD141⁺CLEC9A⁺ cells may be further matured in culture for about 12 to about 21 days, preferably 15 days. The methods provided herein provide for large number of CD141⁺Irf8⁺ cells to be generated. By way of example, and not limitation, approximately 10⁶ CD34⁺ cells may generate about 5×10⁶ to about 5×10⁷ CD141⁺CLEC9A⁺ cDC1 cells in a period of 8-15 days. If needed the cells may be collected after 6-10 days of culturing and reseeded for additional culturing with the FLT3L and GM-CSF for an additional amount of time ranging from 6-14 days. When harvesting the cells adherent cells may be discarded or left behind in the culture containers or plates. The dendritic cells will be non-adherent or semi-adherent. In the Examples, the containers or plates were washed with culture media or saline to remove semi-adherent cells.

The methods provided herein provide for the differentiation of CD34⁺ cells into CD141⁺CLEC9A⁺ cells without the use of feeder cells. Feeder cells are typically a layer of cells unable to divide, which provides extracellular secretions to help another cell to proliferate. It differs from a coculture system because only one cell type is capable of proliferating generally. Feeder cells can be used in stem cell cultures to aid in proliferation and ensure stem cells remain in a pluripotent state. Feeder cells may be irradiated cells and are often fibroblast cells. In addition to maintaining pluripotency, feeder cells also can be used in the differentiation of cells (Balan S. et al, 2018. Cell Report, 24(7), 1902-1915; and Kirkling et al. 2018. Cell Report, 23(12), 3658-3672). Unexpectedly, the methods provided herein allow for the differentiation of CD34⁺ cells into a large number of CD141⁺ CLEC9A⁺ cells without the use of feeder cells. This is advantageous if the end goal is to administer the resultant cells into a human subject by reducing the likelihood of contamination or an adverse immune or other event due to the heterogenous feeder cells.

The methods provided here further provide for the differentiation of CD34⁺ cells into CD141⁺ CLEC9A⁺ cells on sterile untreated culture plates for at least the first 6-8 days of culturing with FLT3L and GM-CSF. Culture plates, tissue culture plates or tissue culture dishes, are sterile plastic plates or containers of many different sizes and configuration but are designed for the growth of cells in vitro. These plates are often made of glass, polystyrene or polypropylene or other types of plastic. Tissue culture plates can be treated to alter the adherence or growth of the cells contained in or cultured in contact with the plate or container. Examples of treated tissue culture plates include but are not limited to tissue-culture treated plates, poly-lysine-coated plates, collagen-coated plates, polyethyleneimine (PEI)-coated plates, streptavidin-coated plates, and antibody-coated plates. Tissue culture treating plates or containers typically includes exposing a polystyrene plate to a plasma gas in order to modify the hydrophobic plastic surface to make it more hydrophilic. The resulting surface carries a net negative charge due to the presence of oxygen-containing functional groups such as hydroxyl and carboxyl. In general, this will lead to increased cell attachment. The methods provided herein comprise the use of non-tissue culture treated plates or containers to culture the CD34⁺ cells and differentiate the cells into DCs. Use of tissue culture plates in the methods described herein results in lower levels of distinguishing cell surface markers including CD141, CLEC9A and TLR3 as compared to using non-tissue culture treated plates.

In some embodiments, CD34⁺ cells may be isolated from a subject with a disease or cancer. The CD34⁺ cells may be differentiated into CD141⁺CLEC9A⁺ cells as described herein and then administered back to the subject. In some embodiments, the differentiated CD141Irf8⁺ cells may be exposed to an antigen prior to administration to the subject. The CD141⁺CLEC9A⁺ cells may be administered in combination with other therapy or standard of care therapeutics. For example, mobilized CD34⁺ may be isolated from a subject with cancer, differentiated into CD141⁺CLEC9A⁺ cells, and administered intratumorally to the subject with cancer. The subject may also be administered an additional immunotherapy or other standard of care therapeutic. Differentiated CD141⁺CLEC9A⁺ cells may be administered more than once to a subject.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

Reference is made to the manuscript: Swartz et al., “The In Vitro Differentiation of Human CD141⁺CLEC9A⁺Dendritic Cells from Mobilized Peripheral Blood CD34⁺Hematopoietic Stem Cells; Current Protocols. 2022” the content of which is incorporated herein by reference in its entirety

### Example 1

Dendritic cells are professional antigen presenting cells that are central to the immune system's ability to detect “non-self” elements and generate adaptive immune responses to pathogens and cancers. As such, they are an attractive immunotherapeutic platform for clinical vaccine studies. Conventional type 1 DCs (cDC1s) represent a DC subset that excels in antigen processing and cytotoxic T-cell activation. In humans, cDC1s are delineated by their expression of the transcription factors Irf8 and BATF3, as well as the surface marker CD141 and other surface markers, such as CLEC9A, TLR3, and XCR1. Unfortunately, their scarcity in human blood has limited their utilization for immunotherapy. Thus, methods that can generate large numbers of cDC1s will be beneficial for their study and application in the clinic.

This protocol describes a method for producing high numbers of CD141⁺Irf8⁺CLEC9A⁺ TLR3⁺ human DCs in vitro. Basic Protocol 1 describes how these DCs are differentiated from mobilized CD34⁺ hematopoietic stem cells using only the cytokines FLT3L and GM-CSF. Supporting Protocol 1 details a method for immunophenotyping cells at various stages of the differentiation protocol by flow cytometry.

Dendritic cells (DCs) are professional antigen presenting cells that serve as the sentinels of the immune system by surveying the environment for “non-self” material and alerting other cells of danger. Under healthy conditions, DCs exist in an immature state where they continuously sample cell-endogenous and exogenous antigen, which are processed through specialized antigen-presentation pathways and presented to CD8⁺ and CD4⁺ T lymphocytes. Upon detection of damaged cells or pathogen-associated motifs by pattern recognition receptors, DCs signal to other cells through the elaboration of various immune-stimulating cytokines and upregulation of several molecules that enhance their T-cell stimulating capabilities—a process known as maturation. Mature DCs are uniquely equipped to be potent activators of naïve T cells. This distinctive ability to bridge the innate and adaptive arms of the immune system makes DCs desirable targets for immunotherapy.

All DCs arise from bone marrow-resident progenitors produced from hematopoietic stem cells. Historically, human DCs have been divided into 3 major subsets: plasmacytoid DCs, conventional type 2 DCs (cDC2), and conventional type 1 DCs (cDC1). Plasmacytoid DCs (pDCs), which can originate from both the common myeloid and common lymphoid progenitor, are typified by the high expression of the toll-like receptors TLR7 and TLR9 (Musumeci, Lutz, Winheim, & Krug, 2019). Upon engagement with endosomal single-stranded RNA and unmethylated CpG present in viral genomes, pDCs secrete large amounts of type I interferons, making these cells instrumental for the antiviral immune response. In contrast, the pre-cDC, which gives rise to both types of conventional DCs, originates only from the common myeloid progenitor. Conventional type 2 DCs specialize in the presentation of exogenous antigen to CD4⁺ T cells and, therefore, play a key role in the response to extracellular pathogens and parasites. Alternatively, cDC1 excel in the presentation of exogenous and cell-intrinsic antigen to CD8⁺ cytotoxic T cells, which are critical for the immune response against intracellular pathogens and cancer (Anderson, Dutertre, Ginhoux, & Murphy, 2021). Recently, single cell transcriptomics have suggested the presence of additional DC subsets (Villani et al., 2017). This includes a cDC2-like Irf81^{low} DC3s, CD141-CD1c-CD16⁺ DC4s, and AXL⁺SIGLEC6⁺ pre-cDC-like DC5s (Cytlak et al., 2020; Dutertre et al., 2019; See et al., 2017). It is currently uncertain, however, whether these are discrete DC subsets or differentiation states of conventional or inflammatory DCs (Cabeza-Cabrerizo, Cardoso, Minutti, Pereira da Costa, & Reis e Sousa, 2021).

An additional DC type that is often included in the DC superfamilies is the monocyte-derived DC (or MoDC). This DC subset is ontogenically distinct from conventional DCs, given their monocytic origins, and emerged upon the finding that human monocytes cultured in vitro in the presence of GM-CSF±IL-4 generate cDC2-like cells (Briseno et al., 2016; Gao et al., 2021; Inaba et al., 1992; Lehtonen et al., 2005). MoDCs quickly became an attractive platform for DC vaccines considering the large numbers of monocytes in blood (˜5-10% of circulating leukocytes); thus, millions of MoDCs can be generated from only a few milliliters of blood. However, despite their ability to induce T-cell activation in vivo, positive clinical outcomes with MoDCs have been modest, at best (Filin, Kitaeva, Rutland, Rizvanov, & Solovyeva, 2021). MoDCs are transcriptionally and phenotypically distinct from naturally occurring DCs (Helft et al., 2015), generate inferior T cell proliferative responses compared to blood-derived DCs (Osugi, Vuckovic, & Hart, 2002), and have suboptimal ability to migrate to draining lymph nodes (Shinde, Fernandes, Melinkeri, Kale, & Limaye, 2018; Verdijk et al., 2009), which may underlie their poor clinical performance.

While MoDCs remain the most common DC type used in clinical studies, several investigators are evaluating natural DC vaccines in an effort to overcome the limitations and disadvantages of MoDCs (Fu, Zhou, Mi, & Jiang, 2020). The first clinical trial using naturally circulating DCs utilized positively enriched BDCA-4⁺ pDCs in patients with metastatic melanoma (Tel et al., 2013). The results from this study demonstrated a type I interferon signature, CD8⁺ and CD4⁺ T-cell responses, and improved overall survival compared to historical controls. Studies using natural CD1c⁺ cDC2s have been conflicting. Two trials indicated no immune response to the targeted antigen using cDC2 (Davis et al., 2017; Prue et al., 2015); however, a 3rd trial, conducted in patients with stage IV metastatic melanoma, revealed that 21% of treated patients experienced a multifunctional CD8⁺ T-cell response, which correlated with progression-free survival—a result that was similar to their earlier findings with MoDCs in a similar setting (Aarntzen et al., 2012; Schreibelt et al., 2016). A more recent trial evaluated naturally circulating cDC2 versus pDC vaccines or a combination of both in patients with advanced prostate cancer. The results indicate that all vaccine formulations were able to induce tumor-specific T-cell responses in >70% of treated patients, with no significant differences between treatment arms (Westdorp et al., 2019). Ultimately, these studies have demonstrated that vaccination with natural DCs is safe, well-tolerated, and feasible despite the low frequency of DCs in the blood (<1% of circulating PBMCs) (Saxena, Balan, Roudko, & Bhardwaj, 2018). However, it is still uncertain whether natural DC vaccines are superior to MoDC vaccines, as no direct comparison has been conducted in a clinical setting.

Natural conventional type 1 DC vaccines have yet to be studied for cancer immunotherapy, likely due to their incredibly low prevalence in the blood. The median yield of blood DCs is 0.69% of PBMCs. Within this population, the median frequencies of pDCs, cDC2s, and cDC1s are 47.6%, 49.4%, and 2.6%, respectively (Hanel, Angerer, Petry, Lichtenegger, & Subklewe, 2021). Thus, cDC1s make up less than 0.03% of human PBMCs (Jongbloed et al., 2010). Given the superiority of cDC1s in generating CD8⁺ T-cell immunity, improved methods for isolating, expanding, or differentiating cDC1s are greatly needed.

Human cDC1 are characterized by the expression CD141 (BDCA-3), CLEC9A, XCR1, and TLR3. Conventional type 1 DC differentiation requires the transcription factor Irf8 (Aliberti et al., 2003), and terminal differentiation requires Irf8 autoactivation by BATF3 (Grajales-Reyes et al., 2015). Their necessity in the antitumor response is evidenced by studies showing loss of spontaneous tumor rejection in mice lacking the BATF3 transcription factor (Broz et al., 2014; Hildner et al., 2008). In mice, cDC1 can be generated from bone marrow cells ex vivo using the cytokines FLT3L and GM-CSF (Mayer et al., 2014). These cells are superior to MoDCs in terms of migrating to draining lymph nodes and inducing antitumor effects (Zhou et al., 2020). Preclinical studies have shown that the injection FLT3L induces the expansion of cDC1 in mice (Hammerich et al., 2019; Salmon et al., 2016); however, FLT3L also expands immunosuppressive Tregs in human subjects (Klein et al., 2013). Thus, FLT3L therapy may not be the most pragmatic way of expanding cDC1s in cancer patients.

In vitro differentiation of CD34⁺ hematopoietic stem cells (HSCs) into DCs represents an attractive means for generating the large numbers of cDC1s required for immunotherapy. Studies have shown that CD34⁺ HSCs can be mobilized into the blood—using G-CSF, Plerixafor, or similar—even in some of the most immune dysfunctional populations, such as individuals with glioblastoma (Adair et al., 2012). Previous methods have demonstrated that CD34⁺ HSCs cultured in the presence FLT3L and other cytokines generate cDC1-like cells; however, these methods produce poor yields or rely on the use of immortalized feeder cells, which could pose challenges for clinical translation (Balan et al., 2018; Balan & Dalod, 2016; Kirkling et al., 2018; Lee et al., 2015; Poulin et al., 2010; Proietto, Mittag, Roberts, Sprigg, & Wu, 2012). The approach outlined in this protocol is capable of generating large numbers of cDC1-like cells using only CD34⁺ HSCs, FLT3L, and GM-CSF.

### Basic Protocol 1

Generation of Human CD141⁺CLEC9A⁺ Dendritic Cells from Mobilized Peripheral Blood CD34⁺ Hematopoietic Stem Cells.

### Materials:

10⁶ G-CSF mobilized human peripheral blood CD34⁺ hematopoietic stem cells obtained from a commercial source (e.g., frozen cells from StemCell Technologies, cat. no. 70060.1 or ZenBio Inc., cat. no. SER-CD34-MPB1-F)
DPBS, no calcium, no magnesium (Gibco, cat. no. 14190144)
RPMI 1640 medium (Gibco, cat. no. 11875085)
Heat inactivated fetal bovine serum (HyClone, cat. no. SH30071.03HI or equivalent)
HEPES (Gibco, cat. no. 15630080)
Sodium pyruvate (Gibco, cat. no. 11360070)
L-glutamine (Gibco, cat. no. 25030081)
2-Mercaptoethanol (Gibco, cat. no. 21985023)
Optional: Penicillin-Streptomycin (Gibco, cat. no. 15140122)
Human FLT3L (Peprotech, cat. no. 300-19-100UG)
Human GM-CSF (Peprotech, cat. no. 300-03-5UG)
Trypan blue solution 0.4% (Gibco, cat. no. 15250061)
Pipet-aid (Drummond, cat. no. 4-000-101 or equivalent)
20 μL, 200 μL, and 1000 μL pipette (Rainin or equivalent)
Sterile 15 and 50 mL conical tubes (Falcon or equivalent)
Sterile 20 μL, 200 μL, and 1000 μL pipette tips (VWR or equivalent)
Sterile 2 mL, 5 mL, 10 mL and 25 mL serological pipettes (Falcon or equivalent)
Sterile 24 and 48 well non-tissue culture treated flat-bottom plates (Olympus or equivalent)
Hemocytometer
Laminar flow hood
Incubator (CO₂ and humidified)
Laboratory microscope (Zeiss or equivalent)

### Day 0—Plating CD34⁺ Hematopoietic Stem Cells (HSCs) for Differentiation Phase

1. Carefully thaw tube containing frozen CD34⁺ HSCs in 37° C. incubator or water bath until only a small amount of ice remains.

We have used G-CSF mobilized CD34⁺ HSCs in all our studies. CD34⁺ HSCs mobilized by other means (e.g., Plerixafor) may also work.

If using fresh CD34⁺ HSCs, proceed to step 3.
2. Carefully transfer CD34⁺ HSCs to a sterile 15 mL conical tube containing 10 mL of R¹⁰ medium using a 1000 μL pipette.

This, and all remaining steps, are to be performed using aseptic technique in a sterile laminar flow hood.
3. Centrifuge CD34⁺ HSCs at 350×g for 10 min at 20° C.
4. Decant supernatant into a waste container and resuspend cells in 2 mL fresh complete CD141 DC medium.
5. Add 10 μL CD34⁺ HSCs to 10 μL 0.4% Trypan blue and enumerate cells by trypan blue exclusion on a hemocytometer.
6. Suspend cells to 4×10⁵ CD34⁺ HSCs/mL and add 500 μL into wells of non-tissue-culture treated 48-well plate.

It is imperative to use non-tissue-culture treated plates for this step. The use of tissue-culture treated plates on day 0 results in Irf8⁺ cells expressing much lower levels of cDC1-specific markers.

We have found that larger and smaller non-tissue culture treated surfaces (e.g., 96-well plate, 24-well plate, 6-well plate, etc.) may be used at this step as long as the plating density remains equivalent to ˜2×10⁵ CD34⁺ HSCs per 1 cm² plating area.
7. Incubate cells at 37° C. in a humidified CO₂ incubator.

### Day 8—Replating Dendritic Cell Progenitors for Maturation Phase

8. Collect non- and semi-adherent cells from the wells of the plate by gentle trituration using a 1000 μL pipette.

The medium within wells may be slightly yellow. In our experience, this does not affect cell viability or yield.
9. Transfer cells to a sterile 15 mL conical tube.
10. Rinse the wells out with sterile DPBS and transfer washes to 15 mL tube containing DC progenitors.
11. Centrifuge cells at 350×g for 10 min at 20° C.
12. Decant supernatant and resuspend cells in 2 mL fresh complete CD141 DC medium.
13. Add 10 μL cells to 40 μL 0.4% Trypan blue and enumerate cells by trypan blue exclusion on a hemocytometer.

There may be some cell debris in sample, but cell viability should be >90%.

From 10⁶ CD34⁺ HSCs plated on day 0, we usually attain 6-8.5×10⁶ viable cells on day 8.

Calculate volume of resuspended cells required to provide 7.6×10⁴ viable cells and pipette this volume into non-tissue-culture treated 24 well plate containing 1 mL fresh complete CD141 DC medium (see Reagents below). During the maturation phase, viable cell numbers should increase ˜7.5-15× by day 15.

Larger or smaller non-tissue culture treated surfaces (e.g., 96-well plate, 48-well plate, 6-well plate, etc.) may be used at this step so long as the plating density remains equivalent to ˜4×10⁴ viable cells per 1 cm² plating area.
14. Incubate cells at 37° C. in a humidified CO₂ incubator.

### Day 15—Dendritic Cell Harvest

15. Harvest non- and semi-adherent cells from all wells by gentle trituration using a 1000 μL pipette.
16. Transfer cells to sterile 15 mL conical tube.
17. Wash residual cells out of wells using sterile DPBS and add to 15 mL tube containing cells.
18. Centrifuge cells at 350×g for 10 min at 20° C.
19. Decant supernatant and resuspend cells in fresh R10 medium.
20. Count cells by trypan blue exclusion with a hemocytometer.

### Support Protocol 1

### Flow Cytometric Immunophenotyping of CD141⁺ Dendritic Cells

The following protocol describes our method for immunophenotyping DCs by flow cytometry. We generally perform this step on day 8 and day 15 of Basic Protocol 1. This protocol is specific for flow cytometers equipped with a plate reader and 405 nm, 488 nm, and 638 nm lasers. Most of the antibody clones used in this support protocol (listed below) are available in a variety of colors from various vendors and can be adapted to other cytometers, as needed.

Materials:
DPBS, no calcium, no magnesium (Gibco, cat. no. 14190144)
Heat inactivated fetal bovine serum (HyClone, cat. no. SH30071.03HI or equivalent
Optional: Transcription factor staining kit (BioLegend, 424401 or similar)
Zombie Green™ Fixable Viability Kit (BioLegend, cat. no. 423111)
   Antibodies for phenotyping (NOTE: choose fluorophores that are compatible with lasers equipped on flow cytometer. The following antibodies, as well as a viability stain in the FITC channel, work with a flow cytometer having a 405 nm laser with 450/45 BP and 780/60 BP filters, a 488 nm laser with 525/40 BP and 585/42 BP filters, and a 638 nm laser with 660/20 BP, 712/25 BP, and 780/60 BP filters):

CD141 surface marker panel: Anti-human CD141-Brilliant Violet™ 785 (BioLegend, cat. no. 344115), anti-human CLEC9A-APC (BioLegend, cat. no. 353805), anti-human CD11c-APC/Fire™ 750 (BioLegend, cat. no. 371509), anti-human TLR3-PE (BioLegend, cat. no. 315009), anti-human CD40-Alexa Fluor® 700 (BioLegend, cat. no. 334327), and anti-human HLA-DR-Brilliant Violet™ 421 (BioLegend, cat. no. 307635).

Optional: Irf8 panel: Anti-human CD141-Brilliant Violet™ 785 (BioLegend, cat. no. 344115), anti-human CLEC9A-APC (BioLegend, cat. no. 353805), anti-human CD11c-APC/Fire™ 750 (BioLegend, cat. no. 371509), anti-human Irf8-PE (BD Biosciences, cat. no. 566373).
Pipet-aid (Drummond, cat. no. 4-000-101 or equivalent)
5 mL serological pipettes (Falcon or equivalent)
2 μL, 20 μL, 200 μL, and 1000 μL pipette (Rainin or equivalent)
2 μL, 20 μL, 200 μL, and 1000 μL pipette tips (VWR or equivalent)
5 mL round bottom tube (Falcon or equivalent)
1.2 mL microfuge tubes (Eppendorf or equivalent)
96-well round bottom plate (Olympus or equivalent)

Flow cytometer (e.g., Beckman Coulter CytoFlex or equivalent)
1. Pipette 5×10⁵ cells into seven 5 mL round bottom tube based on cell counts from step 21 in Basic Protocol 1.

Seven samples will allow for a complete CD141 surface marker stain and 6 fluorescence-minus-one (FMO) controls. If performing the optional Irf8 panel, set up an additional 5 tubes: one for the complete Irf8 stain, and 4 for FMO controls.
2. Bring volume in each round bottom tube up to 4 mL with DPBS
3. Centrifuge cells at 350×g for 7 min at 20° C.
4. Decant supernatant and add 200 μL of a 1:1000 dilution (in DPBS) of Zombie Green viability stain
5. Allow cells to stain in the dark for 15 min at 20° C.
6. Bring volume up to 4 mL with DPBS/2% FBS and incubate for 5 min at 20° C.
7. Centrifuge cells at 350×g for 7 min at 20° C.
8. While cells are centrifuging, create antibody cocktails for surface markers by adding 0.7 μL each antibody to 100 μL DPBS/2% FBS. For FMO controls, each cocktail should omit only 1 fluorophore-conjugated antibody until all fluorophores are accounted for.

The amount of antibody was determined by titration. It may be necessary to titrate each lot of antibody to determine the optimal concentration for staining.

For the complete CD141 surface marker panel, all antibodies are added at this step. For the optional complete Irf8 panel, all antibodies except the Irf8 antibody are added at this step.
9. Decant supernatant and add antibody cocktails to each of the corresponding round bottom tubes containing cells.
10. Allow cells to stain in the dark for 15 min at 20° C.
11. Bring volume up to 4 mL with DPBS/2% FBS
12. Centrifuge cells at 350×g for 7 min at 20° C.
13. Decant supernatant and bring volume to 4 mL with DPBS/2% FBS
14. Centrifuge cells at 350×g for 7 min at 20° C.
15. Decant supernatant

If performing only CD141 surface marker panel, proceed to step 27. Otherwise, store samples at 4° C. until ready to analyze by flow cytometry.
16. Optional step for Irf8 panel: Vortex cells gently, add 1 mL fixation solution to tubes, and incubate at 20° C. for 45 min.
17. Optional step for Irf8 panel: Add 2 mL permeabilization solution to tubes and then centrifuge cells at 350×g for 7 min at 20° C.
18. Optional step for Irf8 panel: Decant supernatant, gently vortex cells, and add 2 mL permeabilization solution to tubes.
19. Optional step for Irf8 panel: Centrifuge cells at 350×g for 7 min at 20° C.
20. Optional step for Irf8 panel: While cells are centrifuging, create staining solution by adding 0.7 μL anti-human Irf8-PE antibody to 100 μL permeabilization solution.
21. Optional step for Irf8 panel: Decant supernatant and add anti-Irf8 staining solution.
22. Optional step for Irf8 panel: Incubate cells in the dark for 30 min at 20° C.
23. Optional step for Irf8 panel: Add 2 mL permeabilization solution to tubes and then centrifuge cells at 350×g for 7 min at 20° C.
24. Optional step for Irf8 panel: Decant supernatant and add 4 mL DPBS/2% FBS.
25. Optional step for Irf8 panel: Centrifuge cells at 350×g for 7 min at 20°.
26. Optional step for Irf8 panel: Decant supernatant.
27. Gently vortex tubes containing cells and transfer cells to wells of a round bottom 96-well plate.
28. Bring volume up to 200 μL with DPBS/2% FBS.
29. Analyze on flow cytometer.

### Reagents and Solutions:

R10 Medium
435 mL RPMI 1640
50 mL FBS (10% v/v final)
5 mL HEPES (10 mM final)
5 mL sodium pyruvate (1 mM final)
5 mL glutamine (2 mM final)
500 μL 2-mercaptoethanol (55 μM final)
Optional: 5 mL Penicillin-Streptomycin (100 U/mL final)
   R10 media may be stored for up to 1 month at 4° C. and warmed to 37° C. in water bath prior to use.

Complete CD141 DC medium
R10 medium
150 ng/mL human FLT3L
10 ng/mL human GM-CSF

Complete CD141 DC medium is made fresh. We prepare aliquots of human FLT3L and GM-CSF that can make up 5 mL and 25 mL of complete CD141 DC medium to mitigate waste of cytokines.

### Troubleshooting:

The use of non-tissue culture treated plates on day 0 is a key difference between the methods provided here and the prior methods used in mouse models. We have attempted using tissue-culture treated plates on day 0, and while the resulting cells are still Irf8⁺, they do not express high levels of CD141, CLEC9A, and TLR3. Splitting cells into tissue-culture treated plates on day 8 of Basic Protocol 1 does tend to improve yield but at the expense of slightly lower expression of CD141, CLEC9A, and TLR3.

Care should be taken when collecting cells on day 8 and 15 of Basic Protocol 1 to harvest only the non- and semi-adherent fraction, as these will be enriched with DC and DC progenitors. To avoid detaching adherent cells, gentle trituration using a 1000 μL pipette or serological pipette controller on the lowest setting should be used. Additional washes with room temperature PBS may also be used to increase yield.

Reagents can vary among vendors, and this may alter outcomes from DC differentiation protocols. For this protocol, we have used only frozen G-CSF mobilized, peripheral blood CD34⁺ HSCs purchased from StemCell Technologies and ZenBio, Inc. Both vendors' cells yielded similar results with no noticeable differences among donors. Per the manufacturers' descriptions, CD34⁺ HSCs are isolated from peripheral blood using immunomagnetic positive selection. These cells were stored at less than −130° C. for up to 6 months prior to use, with no adverse effects on outcome. Viabilities of CD34⁺ HSCs were always at least 80% and purity greater than 90% on the day of plating. Cytokines sourced from different vendors may also exhibit differential activity. We have used only Peprotech GM-CSF and FLT3L and routinely achieve consistent results. Nevertheless, it is advisable to test reagents in a small batch before proceeding with a large-scale differentiation.

Cell viability should remain high throughout the duration of this protocol (>85%). Quality control of DC and DC progenitors should be performed flow cytometrically using the method outlined in Supporting Protocol 1. While the expression of several of the cDC1 markers will be low at the early stages of this differentiation protocol, CD141 and Irf8 expression starts early and remains high, and thus should be used to monitor cDC1 differentiation.

### Understanding Results:

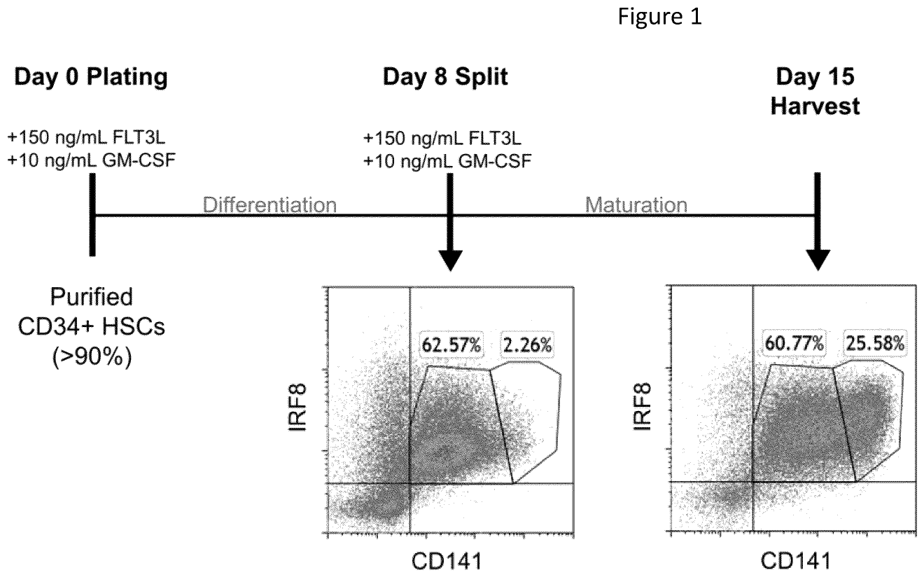

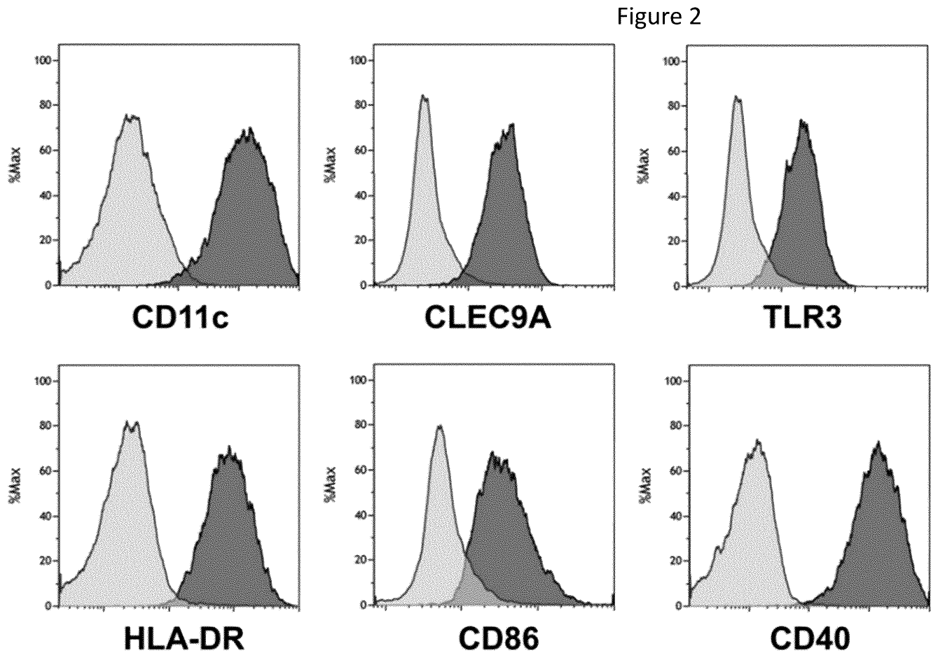

FIGS. 1 and 2 illustrate the expected results from this protocol. In our experience, CD141⁺Irf8⁺ DCs constitute >50% of all viable cells on day 8 and >70% of viable cells on day 15. Irf8⁺ DCs expressing high levels of CD141 co-express high levels of several cDC1 and maturation markers; these cells make up ˜25% of all viable cells on day 15. Irf8⁺ DCs expressing moderate levels of CD141 also express cDC1 and maturation markers (not shown), albeit at lower levels than CD141^{HI} DCs. Expected viable cell yields on day 8 of this protocol are ˜1.10-1.55×10⁶ cells per 1 cm² plating area on day 0 and day 15 yields are ˜3-6×10⁵ cells per 1 cm² plating area on day 8. Thus, from 10⁶ CD34⁺ HSCs plated on day 0, it is reasonable to generate 1-3×10⁷ CD11c⁺Irf8⁺CD141^{HI}CLEC9A⁺ TLR3⁺DCs on day 15 using this protocol.

### Time Considerations:

In Basic Protocol 1, 1-3 hours are needed to prepare CD141 medium and to wash, enumerate, and plate CD34⁺ HSCs on day 0. The replating step on day 8 and harvesting step on day 15 require 30-90 min of processing time. Immunophenotyping cells may take 1-2 hours for extracellular staining only and 4-6 hours if performing the optional Irf8⁺staining protocol in Supporting Protocol 1.

### REFERENCES

Aarntzen, E. H., Bol, K., Schreibelt, G., Jacobs, J. F., Lesterhuis, W. J., Van Rossum, M. M., . . . De Vries, I. J. (2012). Skin-test infiltrating lymphocytes early predict clinical outcome of dendritic cell-based vaccination in metastatic melanoma. Cancer Res, 72(23), 6102-6110. doi:10.1158/0008-5472.CAN-12-2479
Adair, J. E., Beard, B. C., Trobridge, G. D., Neff, T., Rockhill, J. K., Silbergeld, D. L., . . . Kiem, H. P. (2012). Extended survival of glioblastoma patients after chemoprotective HSC gene therapy. Sci Transl Med, 4(133), 133ra157. doi:10.1126/scitranslmed.3003425
Aliberti, J., Schulz, O., Pennington, D. J., Tsujimura, H., Reis e Sousa, C., Ozato, K., & Sher, A. (2003). Essential role for ICSBP in the in vivo development of murine CD8alpha⁺ dendritic cells. Blood, 101(1), 305-310. doi:10.1182/blood-2002-04-1088
Anderson, D. A., 3rd, Dutertre, C. A., Ginhoux, F., & Murphy, K. M. (2021). Genetic models of human and mouse dendritic cell development and function. Nat Rev Immunol, 21(2), 101-115. doi:10.1038/s41577-020-00413-x
Balan, S., Arnold-Schrauf, C., Abbas, A., Couespel, N., Savoret, J., Imperatore, F., . . . Dalod, M. (2018). Large-Scale Human Dendritic Cell Differentiation Revealing Notch-Dependent Lineage Bifurcation and Heterogeneity. Cell Rep, 24(7), 1902-1915 e1906. doi:10.1016/j.celrep.2018.07.033
Balan, S., & Dalod, M. (2016). In Vitro Generation of Human XCR1(⁺) Dendritic Cells from CD34(⁺) Hematopoietic Progenitors. Methods Mol Biol, 1423, 19-37. doi:10.1007/978-1-4939-3606-9_2
Briseno, C. G., Haldar, M., Kretzer, N. M., Wu, X., Theisen, D. J., Kc, W., . . . Murphy, K. M. (2016). Distinct Transcriptional Programs Control Cross-Priming in Classical and Monocyte-Derived Dendritic Cells. Cell Rep, 15(11), 2462-2474. doi:10.1016/j.celrep.2016.05.025
Broz, M. L., Binnewies, M., Boldajipour, B., Nelson, A. E., Pollack, J. L., Erle, D. J., . . . Krummel, M. F. (2014). Dissecting the tumor myeloid compartment reveals rare activating antigen-presenting cells critical for T cell immunity. Cancer Cell, 26(5), 638-652. doi:10.1016/j.ccell.2014.09.007
Cabeza-Cabrerizo, M., Cardoso, A., Minutti, C. M., Pereira da Costa, M., & Reis e Sousa, C. (2021). Dendritic Cells Revisited. Annu Rev Immunol, 39, 131-166. doi:10.1146/annurev-immunol-061020-053707
Cytlak, U., Resteu, A., Pagan, S., Green, K., Milne, P., Maisuria, S., . . . Bigley, V. (2020). Differential IRF8 Transcription Factor Requirement Defines Two Pathways of Dendritic Cell Development in Humans. Immunity, 53(2), 353-370 e358. doi:10.1016/j.immuni.2020.07.003
Davis, I. D., Quirk, J., Morris, L., Seddon, L., Tai, T. Y., Whitty, G., . . . Cebon, J. (2017). A pilot study of peripheral blood BDCA-1 (CD1c) positive dendritic cells pulsed with NY-ESO-1 ISCOMATRIX adjuvant. Immunotherapy, 9(3), 249-259. doi:10.2217/imt-2016-0132
Dutertre, C. A., Becht, E., Irac, S. E., Khalilnezhad, A., Narang, V., Khalilnezhad, S., . . . Ginhoux, F. (2019). Single-Cell Analysis of Human Mononuclear Phagocytes Reveals Subset-Defining Markers and Identifies Circulating Inflammatory Dendritic Cells. Immunity, 51(3), 573-589 e578. doi:10.1016/j.immuni.2019.08.008
Filin, I. Y., Kitaeva, K. V., Rutland, C. S., Rizvanov, A. A., & Solovyeva, V. V. (2021). Recent Advances in Experimental Dendritic Cell Vaccines for Cancer. Front Oncol, 11, 730824. doi:10.3389/fonc.2021.730824
Fu, C., Zhou, L., Mi, Q. S., & Jiang, A. (2020). DC-Based Vaccines for Cancer Immunotherapy. Vaccines (Basel), 8(4). doi:10.3390/vaccines8040706
Gao, Y., Li, H., Li, Z., Xie, L., Liu, X., Huang, Z., . . . Su, W. (2021). Single-Cell Analysis Reveals the Heterogeneity of Monocyte-Derived and Peripheral Type-2 Conventional Dendritic Cells. J Immunol, 207(3), 837-848. doi:10.4049/jimmunol.2100094
Grajales-Reyes, G. E., Iwata, A., Albring, J., Wu, X., Tussiwand, R., Kc, W., . . . Murphy, K. M. (2015). Batf3 maintains autoactivation of Irf8 for commitment of a CD8alpha(⁺) conventional DC clonogenic progenitor. NatImmunol, 16(7), 708-717. doi:10.1038/ni.3197
Hammerich, L., Marron, T. U., Upadhyay, R., Svensson-Arvelund, J., Dhainaut, M., Hussein, S., . . . Brody, J. D. (2019). Systemic clinical tumor regressions and potentiation of PD1 blockade with in situ vaccination. Nat Med, 25(5), 814-824. doi:10.1038/s41591-019-0410-x
Hanel, G., Angerer, C., Petry, K., Lichtenegger, F. S., & Subklewe, M. (2021). Blood DCs activated with R848 and poly(I:C) induce antigen-specific immune responses against viral and tumor-associated antigens. Cancer ImmunolImmunother. doi:10.1007/s00262-021-03109-w
Helft, J., Bottcher, J., Chakravarty, P., Zelenay, S., Huotari, J., Schraml, B. U., . . . Reis e Sousa, C. (2015). GM-CSF Mouse Bone Marrow Cultures Comprise a Heterogeneous Population of CD11c(⁺)MHCII(⁺) Macrophages and Dendritic Cells. Immunity, 42(6), 1197-1211. doi:10.1016/j.immuni.2015.05.018
Hildner, K., Edelson, B. T., Purtha, W. E., Diamond, M., Matsushita, H., Kohyama, M., . . . Murphy, K. M. (2008). Batf3 deficiency reveals a critical role for CD8alpha⁺ dendritic cells in cytotoxic T cell immunity. Science, 322(5904), 1097-1100. doi:10.1126/science.1164206 Inaba, K., Inaba, M., Romani, N., Aya, H., Deguchi, M., Ikehara, S., . . . Steinman, R. M. (1992). Generation of large numbers of dendritic cells from mouse bone marrow cultures supplemented with granulocyte/macrophage colony-stimulating factor. J Exp Med, 176(6), 1693-1702. doi:10.1084/jem.176.6.1693
Jongbloed, S. L., Kassianos, A. J., McDonald, K. J., Clark, G. J., Ju, X., Angel, C. E., . . . Radford, K. J. (2010). Human CD141⁺(BDCA-3)⁺ dendritic cells (DCs) represent a unique myeloid DC subset that cross-presents necrotic cell antigens. J Exp Med, 207(6), 1247-1260. doi:10.1084/jem.20092140
Kirkling, M. E., Cytlak, U., Lau, C. M., Lewis, K. L., Resteu, A., Khodadadi-Jamayran, A., . . . Reizis, B. (2018). Notch Signaling Facilitates In Vitro Generation of Cross-Presenting Classical Dendritic Cells. Cell Rep, 23(12), 3658-3672 e3656. doi:10.1016/j.celrep.2018.05.068 Klein, O., Ebert, L. M., Zanker, D., Woods, K., Tan, B. S., Fucikova, J., . . . Cebon, J. (2013). Flt3 ligand expands CD4⁺ FoxP3⁺ regulatory T cells in human subjects. Eur J Immunol, 43(2), 533-539. doi:10.1002/eji.201242603
Lee, J., Breton, G., Oliveira, T. Y., Zhou, Y. J., Aljoufi, A., Puhr, S., . . . Liu, K. (2015). Restricted dendritic cell and monocyte progenitors in human cord blood and bone marrow. J Exp Med, 212(3), 385-399. doi:10.1084/jem.20141442
Lehtonen, A., Veckman, V., Nikula, T., Lahesmaa, R., Kinnunen, L., Matikainen, S., & Julkunen, I. (2005). Differential expression of IFN regulatory factor 4 gene in human monocyte-derived dendritic cells and macrophages. J Immunol, 175(10), 6570-6579. doi:10.4049/jimmunol.175.10.6570
Mayer, C. T., Ghorbani, P., Nandan, A., Dudek, M., Arnold-Schrauf, C., Hesse, C., . . . Sparwasser, T. (2014). Selective and efficient generation of functional Batf3-dependent CD103⁺dendritic cells from mouse bone marrow. Blood, 124(20), 3081-3091. doi:10.1182/blood-2013-12-545772
Musumeci, A., Lutz, K., Winheim, E., & Krug, A. B. (2019). What Makes a pDC: Recent Advances in Understanding Plasmacytoid DC Development and Heterogeneity. Front Immunol, 10, 1222. doi:10.3389/fimmu.2019.01222
Osugi, Y., Vuckovic, S., & Hart, D. N. (2002). Myeloid blood CD11c(⁺) dendritic cells and monocyte-derived dendritic cells differ in their ability to stimulate T lymphocytes. Blood, 100(8), 2858-2866. doi:10.1182/blood.V100.8.2858
Poulin, L. F., Salio, M., Griessinger, E., Anjos-Afonso, F., Craciun, L., Chen, J. L., . . . Reis e Sousa, C. (2010). Characterization of human DNGR-1⁺ BDCA3⁺ leukocytes as putative equivalents of mouse CD8alpha⁺ dendritic cells. J Exp Med, 207(6), 1261-1271. doi:10.1084/jem.20092618
Proietto, A. I., Mittag, D., Roberts, A. W., Sprigg, N., & Wu, L. (2012). The equivalents of human blood and spleen dendritic cell subtypes can be generated in vitro from human CD34(⁺) stem cells in the presence of fms-like tyrosine kinase 3 ligand and thrombopoietin. Cell Mol Immunol, 9(6), 446-454. doi:10.1038/cmi.2012.48
Prue, R. L., Vari, F., Radford, K. J., Tong, H., Hardy, M. Y., D'Rozario, R., . . . Hart, D. N. (2015). A phase I clinical trial of CD1c (BDCA-1)⁺ dendritic cells pulsed with HLA-A*0201 peptides for immunotherapy of metastatic hormone refractory prostate cancer. J Immunother, 38(2), 71-76. doi:10.1097/CJI.0000000000000063
Salmon, H., Idoyaga, J., Rahman, A., Leboeuf, M., Remark, R., Jordan, S., . . . Merad, M. (2016). Expansion and Activation of CD103(⁺) Dendritic Cell Progenitors at the Tumor Site Enhances Tumor Responses to Therapeutic PD-L1 and BRAF Inhibition. Immunity, 44(4), 924-938. doi:10.1016/j.immuni.2016.03.012
Saxena, M., Balan, S., Roudko, V., & Bhardwaj, N. (2018). Towards superior dendritic-cell vaccines for cancer therapy. Nat Biomed Eng, 2(6), 341-346. doi:10.1038/s41551-018-0250-x Schreibelt, G., Bol, K. F., Westdorp, H., Wimmers, F., Aarntzen, E. H., Duiveman-de Boer, T., . . .
de Vries, I. J. (2016). Effective Clinical Responses in Metastatic Melanoma Patients after Vaccination with Primary Myeloid Dendritic Cells. Clin Cancer Res, 22(9), 2155-2166. doi:10.1158/1078-0432.CCR-15-2205
See, P., Dutertre, C. A., Chen, J., Gunther, P., McGovern, N., Irac, S. E., . . . Ginhoux, F. (2017). Mapping the human DC lineage through the integration of high-dimensional techniques. Science, 356(6342). doi:10.1126/science.aag3009
Shinde, P., Fernandes, S., Melinkeri, S., Kale, V., & Limaye, L. (2018). Compromised functionality of monocyte-derived dendritic cells in multiple myeloma patients may limit their use in cancer immunotherapy. Sci Rep, 8(1), 5705. doi:10.1038/s41598-018-23943-w Tel, J., Aarntzen, E. H., Baba, T., Schreibelt, G., Schulte, B. M., Benitez-Ribas, D., . . . de Vries,
I. J. (2013). Natural human plasmacytoid dendritic cells induce antigen-specific T-cell responses in melanoma patients. Cancer Res, 73(3), 1063-1075. doi:10.1158/0008-5472.CAN-12-2583
Verdijk, P., Aarntzen, E. H., Lesterhuis, W. J., Boullart, A. C., Kok, E., van Rossum, M. M., . . . de Vries, I. J. (2009). Limited amounts of dendritic cells migrate into the T-cell area of lymph nodes but have high immune activating potential in melanoma patients. Clin Cancer Res, 15(7), 2531-2540. doi:10.1158/1078-0432.CCR-08-2729
Villani, A. C., Satija, R., Reynolds, G., Sarkizova, S., Shekhar, K., Fletcher, J., . . . Hacohen, N. (2017). Single-cell RNA-seq reveals new types of human blood dendritic cells, monocytes, and progenitors. Science, 356(6335). doi:10.1126/science.aah4573
Westdorp, H., Creemers, J. H. A., van Oort, I. M., Schreibelt, G., Gorris, M. A. J., Mehra, N., . . . Gerritsen, W. R. (2019). Blood-derived dendritic cell vaccinations induce immune responses that correlate with clinical outcome in patients with chemo-naive castration-resistant prostate cancer. J Immunother Cancer, 7(1), 302. doi:10.1186/s40425-019-0787-6
Zhou, Y., Slone, N., Chrisikos, T. T., Kyrysyuk, O., Babcock, R. L., Medik, Y. B., . . . Watowich, S. S. (2020). Vaccine efficacy against primary and metastatic cancer with in vitro-generated CD103(⁺) conventional dendritic cells. J Immunother Cancer, 8(1). doi:10.1136/jitc-2019-000474

### Example 2

In this example several key functions of cDC1s were assessed with in vitro-derived CD141+CLEC9+ DCs. Hallmark features of cDC1s include enhanced ability to cross present exogenous antigen and responsiveness to TLR3 agonism.

Antigen cross presentation is the ability to take up extracellular antigen, digest the antigen into its constituent epitopes (MHC-binding portions of an antigen), load MHC I-restricted epitopes onto endogenous MHC I molecules, and present said MHC I epitopes to CD8+ T cells. Among all of the DC subsets, cDC1 are specialized in this ability, though other DCs subsets, including MoDCs, can carry out this function to a lesser degree. To evaluate antigen cross presentation, 10⁴ in vitro-derived human CD141+CLEC9+ DCs or MoDCs from an HLA-A*0201 donor were resuspended in serum-free AIM V medium and co-incubated with varying concentrations of cytomegalovirus pp65 antigen (Abcam) or no protein (negative control) for 3 hours in a V-bottom plate (Costar). CMV pp65 antigen concentrations tested include 10 ng/mL, 100 ng/mL, 1 μg/mL, and 10 μg/mL. Cells were washed 3 times with sterile PBS to remove residual extracellular pp65 antigen and resuspended in fresh AIM V medium. 5×10⁴ human CD8+ T cells, reactive to the CMV pp65 HLA-A*0201 epitope NLVPMVATV (SEQ ID NO: 1), were added to all wells containing DCs, and co-cultures were incubated for 20 hours at 37° C./5% CO₂. Cells were pelleted by centrifugation (350g×5 min) and supernatant carefully removed for analysis by human IFNγ ELISA (BioLegend).

CD141+CLEC9+ DCs required lower concentrations of antigen in order to stimulate CD8+ T cells compared to MoDCs, indicating superiority in antigen cross-presentation ability (FIG. 3). Stimulation of pp65-cognate CD8+ T cells, as determined by IFNγ secretion, by pp65-loaded CD141+CLEC9+ DCs was evident at concentrations as low as 100 ng/mL. MoDCs, however, required a 10-fold higher concentration of pp65 antigen to stimulate pp65-cognate CD8+ T cells to a similar degree.

Flow cytometric analysis of CD141+CLEC9+ DCs revealed high expression of TLR3 (FIG. 2). TLR3 is uniquely expressed on cDC1 and is rarely found on other DC subsets. TLR3 expression enables cells to respond to extracellular double-stranded RNA, often found in some viral genomes. To confirm that TLR3 on CD141+CLEC9+ DCs was functional, cells were stimulated with the dsRNA analogue polyinosinic-polycytidylic acid (i.e., poly(I:C)) and cytokine secretion assessed using a multiplex ELISA (BioLegend).

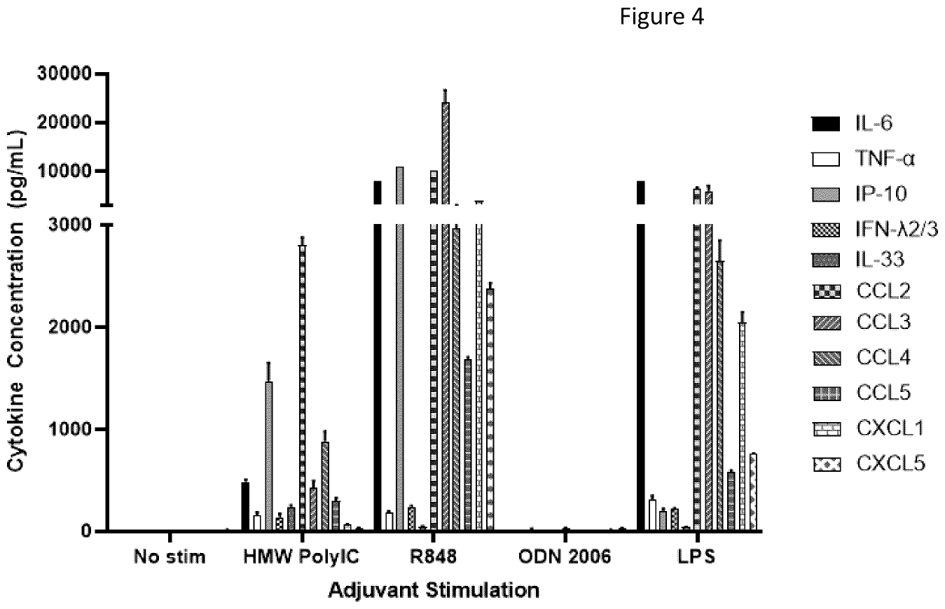

CD141+CLEC9+ DCs stimulated with polyL:C produced high levels of the T-cell chemoattractant CXCL10 (IP-10), confirming that in vitro-derived CD141+CLEC9+ DCs express functional TLR3 (FIG. 4). Additionally, CD141+CLEC9+ DCs also responded to TLR agonists R848 and LPS but no ODN 2006, suggesting CD141+CLEC9+ DCs also express TLR7/8 and TLR4 but not TLR9, respectively.

1. A population of in vitro or ex vivo derived dendritic cells, comprising at least 1×10⁶ dendritic cells/ml, wherein the dendritic cells are CD141⁺CLEC9A⁺.

2. The population of claim 1, wherein the dendritic cells comprise an antigen or nucleic acid added to the cells in vitro or ex vivo.

3. (canceled)

4. The population of in vitro or ex vivo derived dendritic cells of claim 2, wherein the antigen is a peptide or wherein the nucleic acid is an mRNA polynucleotide or a construct encoding a mRNA polynucleotide and the mRNA or construct encoding mRNA is transfected or electroporated into the dendritic cell.

5. The population of claim 2, wherein the antigen is a viral, bacterial, fungal, or tumor antigen.

6. (canceled)

7. The population of claim 1, wherein the in vitro or ex vivo derived dendritic cells activate CD8⁺ T cells.

8.-10. (canceled)

11. A method of treating a subject having a disease, wherein the disease is a viral infection, bacterial infection, fungal infection or cancer, or a disease wherein a Th1 immune response would be beneficial to the subject, the method comprising administering to the subject the population of cells of claim 1.

12. The method of claim 11, wherein the subject has cancer, and the population of cells are administered intratumorally.

13. A method of treating a subject having a disease, wherein the disease is a viral infection, bacterial infection, fungal infection or cancer, or a disease wherein a Th1 immune response would be beneficial to the subject, the method comprising administering to the subject the population of cells of claim 2, wherein the antigen is a peptide, mRNA or construct encoding an mRNA and the cells are administered intravenously, intramuscularly, subcutaneously, intradermally or intratumorally.

14. (canceled)

15. The method of claim 11, wherein the population of dendritic cells are differentiated from CD34⁺ hematopoietic stem cells in a cell culture media comprising FLT3L and GM-CSF.

16. (canceled)

17. The method of claim 15, wherein the population of dendritic cells are produced on sterile untreated polystyrene culture plates.

18. The method of claim 11, wherein CD34⁺ hematopoietic stem cells are mobilized and collected from the subject, differentiated into the population of dendritic cells prior to being administered to the subject to provide a population of autologous dendritic cells to the subject.

19. (canceled)

20. A method of enhancing an immune response in a subject, comprising administering to a subject the population of dendritic cells of claim 1.

21. (canceled)

22. The method of claim 20, wherein the immune response is directed to a viral infection, bacterial infection, fungal infection, cancer or is a Th1 immune response.

23.-24. (canceled)

25. The method of claim 20, wherein the population of dendritic cells are differentiated from CD34⁺ hematopoietic stem cells in a cell culture media comprising FLT3L and GM-CSF.

26. (canceled)

27. The method of claim 25, wherein the population of dendritic cells are produced on sterile untreated culture plates.

28. The method of claim 25, wherein CD34⁺ hematopoietic stem cells are mobilized and collected from the subject, differentiated into the population of dendritic cells prior to being administered to the subject to provide a population of autologous dendritic cells to the subject.

29. The method of claim 11, additionally comprising administering immune checkpoint inhibitor therapy.

30. (canceled)

31. A method of generating a population of human CD141⁺CLEC9A⁺ dendritic cells, the method comprising:
(a) harvesting or obtaining CD34⁺ hematopoietic stem cells from the peripheral blood of a subject;
(b) culturing the CD34⁺ hematopoietic stem cells in non-tissue culture treated containers at a plating density of 1-3×10⁵ cells/cm² in cell culture media comprising FLT3L and GM-CSF to allow the cells to differentiate into cDC1 cells;
(c) collecting the CD141⁺CLEC9A⁺ dendritic cells.

32. The method of claim 31, wherein the subject was treated with G-CSF and/or Plerixafor to mobilize CD34⁺ hematopoietic stem cells into the peripheral blood at least one day prior to harvesting.

33. (canceled)

34. The method of claim 31, wherein the subject is in need of treatment with a cDC1 cell-based therapy.

35. The method of claim 31, wherein 50-250 ng/mL of human FLT3L is added to the cell culture media in step (b) and wherein 1-20 ng/mL of human GM-CSF is added to the cell culture media in step (b).

36.-37. (canceled)

38. The method of claim 31, wherein the cells harvested in step (c) are non-adherent or semi-adherent cells.


SEQUENCE LISTING (1 sequence)

SEQ ID NO: 1 - protein, 9 aa, synthetic construct
NLVPMVATV